(19) United States
(12) Patent Application Publication
(10) Pub. No.: US 2020/0101100 A1
(43) Pub. Date: Apr. 2, 2020

(54) Transgene-Cytotoxic Combination Therapy

(71) Applicant: Gliotherapy Limited, Bladon (GB)

(72) Inventors: Haritha SAMARANAYAKE, Kuopio (FI); Seppo YLA-HERTTUALA, Kuopio (FI); Ann-Marie MAATTA, Kuopio (FI); Jere KURKIPURO, Kuopio (FI)

(73) Assignee: Gliotherapy Limited, Bladon (GB)

(21) Appl. No.: 16/663,649

(22) Filed: Oct. 25, 2019

Related U.S. Application Data
(63) Continuation of application No. 13/877,246, filed on Aug. 7, 2013.

(30) Foreign Application Priority Data
Jan. 18, 2011 (GB) GB11/00804.2
Jan. 18, 2012 (GB) PCT/GB2012/050108

(51) Int. Cl. A61K 31/7088; A61K 38/45; A61K 48/00; C12N 9/12; A61K 31/495; A61K 31/522; A61K 45/06

(52) U.S. Cl. CPC A61K 31/7088; A61K 38/45; A61K 48/0083; A61K 45/06; C12Y 207/01021; A61K 31/495; A61K 31/522; C12N 9/1211

(57) ABSTRACT

A viral gene therapy combination therapy for cancer, using a virus to deliver a gene to a human patient, where the gene expresses a polypeptide which is not native to the wild-type virus, yet which is nonetheless therapeutically useful when administered in combination with a chemotherapeutic agent.

### RELATED APPLICATIONS

This application is a Continuation of co-pending U.S. patent application Ser. No. 13/877,246 filed 7 Aug. 2013, which is the US National Stage entry of Patent Cooperation Treaty application Serial No. PCT/GB2012/050108 filed 18 Jan. 2012, which asserts priority from Great Britain patent application Serial No. GB11/00804.2 filed 18 Jan. 2011, the contents of which are here incorporated by reference.

### UNITED STATES GOVERNMENT INTEREST

None

### FIELD

This invention relates to a drug combination for the treatment of cancer or of a disease characterised by an impaired mismatch repair (MMR) pathway.

### BACKGROUND

Herpes simplex virus type 1, thymidine kinase (HSV-tk) gene therapy is based on the prodrug activating enzyme that converts a non-toxic compounds such as ganciclovir, (GCV) into a toxic metabolite. The cell destruction by HSV-tk/GCV is cell cycle dependent, where only dividing cells will be affected. This is of particular advantage in brain cancer gene therapy, where the rapidly dividing tumor cells are surrounded by non-dividing normal brain cells. Therapy by HSV-tk is disclosed in EP1 135513.

Temozolomide (TMZ, imidazole tetrazinone) is an oral alkylating agent that can cross the blood brain barrier (BBB). Temozolomide is an oral alkylating agent that is a derivative of dacarbazine. TMZ undergoes spontaneous hydrolysis at physiological pH to its active form 3-methyl-(triazen-1-yl) imidazole-4 carboxyamide (MTIC). The primary mode of cytotoxicity is by adding a methyl group at Opposition of guanine (O⁶-mG).

O⁶-mG by itself is not toxic to the cells. However, O⁶-mGs will become cytotoxic as a result of repeated cycles of futile efforts at repair by mismatch repair (MMR) pathway. This will ultimately lead to DNA strand breaks. It is known that a functional MMR pathway is essential to make cells sensitive to TMZ, in the absence of an active MGMT repair pathway (which occurs in 50% of malignant gliomas). Furthermore, defects in the MMR pathway can contribute to almost 100-fold resistance to alkylating agents such as TMZ.

### SUMMARY

The present invention is based on the discovery that HSV-tk gene therapy increases the gene expression of key mismatch repair (MMR) pathway proteins, namely MSH2 and MLH1. This led to the finding that HSV-tk/GCV gene therapy sensitizes cells to chemotherapeutic agents, such as temozolomide (TMZ).

A study designed by the inventors confirmed that a combination of vector/prodrug gene therapy (such as HSV-tk/GCV) and a cytotoxic agent, has much improved efficacy in certain diseases (cancer was tested, but it is believed that this applies to all diseases characterized by an impaired MMR pathway), when compared to the use of either of the components alone, i.e. chemotherapy or vector/prodrug gene therapy.

It was also found that the administration protocol of these components is key to the surprising technical effect observed in the invention, i.e. the synergy. The inventors have found that the upregulation of the MMR pathway by vector/prodrug gene therapy takes approximately 2 days, and lasts for a maximum of 7 days after stopping prodrug therapy. Therefore, in order to see synergy it is necessary to begin administering the cytotoxic agent no later than 7 days after finishing prodrug therapy.

Furthermore, when the condition to be treated is characterised by an impaired MMR pathway, it is believed that a therapeutic benefit may be achieved by administering only the vector/prodrug gene therapy.

In a first aspect, the present invention is characterised by a new dosage regimen. Therefore, according to a first aspect, the present invention is an agent comprising a vector having a functional gene, a prodrug which can be converted into a cytotoxic agent by an expression product of the gene, and another cytotoxic agent, as a combined preparation for simultaneous, sequential or separate use in the therapy of cancer or of a disease characterised by an impaired mismatch repair (MMR) pathway, wherein the dosage regimen comprises beginning prodrug therapy after the vector has been administered, and beginning the another cytotoxic agent therapy no later than 7 days after the prodrug therapy has finished.

According to a second aspect, the present invention is an agent comprising a vector having a functional gene, and a prodrug which can be converted into a cytotoxic agent by an expression product of the gene, as a combined preparation for simultaneous, sequential or separate use in the therapy of a disease characterised by an impaired mismatch repair (MMR) pathway.

According to a third aspect, a method of treating glioblastoma multiforme, comprises the steps of:

a. Diagnosing in a human patient glioblastoma multiforme;

b. Identifying in said patient at least one glioblastoma multiforme tumor;

c. Resectioning said glioblastoma multiforme tumor to remove at least part of said glioblastoma multiforme tumor and expose tumor bed tissue;

d. Administering to said tumor bed tissue an AdHSV-i/c adenoviral vector having a gene coding for thymidine kinase, whereby said AdHSV-i/c adenoviral vector transfects said tumor bed tissue and said tumor bed tissue expresses said gene coding for thymidine kinase;

e. Within about 5 to about 19 days after administering said adenoviral vector to said human patient, further administering to said human patient ganciclovir;

f. Administering to said human patient temozolomide per os or by intravenous infusion.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1 and 2 show mean tumor volume at days 28 and 42 for different HSV-tk/GCV and TMZ dosage regimens.

FIG. 3 shows survival rate for different HSV-tk/GCV and TMZ dosage regimens.

### DETAILED DESCRIPTION

The present invention requires the administration of a vector having a functional gene, and a prodrug which can be converted by an expression product of that gene, into a cytotoxic agent. Preferably, the functional gene is a functional thymidine kinase gene. Preferably, the prodrug is ganciclovir or its analogues. It will be understood that the prodrug therapy should commence after the vector has been administered. Preferably the prodrug is administered from 5 to 19 days after administration of the vector.

Alternatively, suicide genes such as cytosine deminase, cytochrome P450, E coli purine nucleoside phosphorylase and carboxypeptidase G2, are suitable for use in the invention. Those suicide genes can be used in combination with suitable prodrugs, such as 5-fluorocytosine, cyclophosphamide, 6-methylepurine or F-araAMP or 4-benzoyl-L-glutamic acid (CMDA) or their chemical analogs, respectively. In one embodiment, the suicide gene, i.e. the vector, is cytosine deminase, and the prodrug is 5-fluorocytosine is suitable for use in the invention.

The vector is preferably locally administrated. When the therapy is of a cancerous tumor, for example, the vector may be administered directly into that cancerous tumor. Alternatively, it may be preferable to surgically remove the cancerous tumor, and then administer the vector into the wall of the tumor cavity.

As used herein, the term “wall of the tumor cavity” means the area of apparently healthy tissue (i.e., tissue which is apparently healthy to the eye of a surgeon) that remains once a tumor (or part of that tumor) is removed. Although the tissue is apparently healthy, it may contain malignant cells. The term “wall of the tumor cavity” refers to an area of non-tumor mass.

Preferably, the tumor resection is complete as possible, i.e. more than 90%, 95% or 98%. In a preferred embodiment, the vector is administered by injection approximately 1 cm (preferably between 0.5 cm and 5 cm, more preferably between 0.8 cm and 3 cm) deep into the wall of the tumor cavity. This ensures that the vector is into healthy tissue, i.e. is targeting primarily healthy cells (although it is appreciated that some malignant cells may reside in that area of apparently healthy tissue).

The vector that is used to transfer the gene may be any viral vector. However, it is preferred that it is derived from an adenovirus or a lentivirus. More preferably, it is derived from adenovirus.

The present invention is a combination therapy, comprising the administration of a gene therapy vector, a prodrug and a cytotoxic agent. The cytotoxic agent is preferably different from the cytotoxic agent that results from conversion of the prodrug (for example conversion of the ganciclovir), but otherwise the exact nature of the cytotoxic agent is not crucial, but it should preferably be a drug whose function is impaired by impaired MMR pathway. Some preferred cytotoxic agents are:

a) a chloroethylating agents such as carmustine, lomustine, fotemustine, nimustine, ranimustine or streptozocin;

b) a non-classical alkylating agent such as procarbazine;

c) a methylating triazine such as temozolomide, dacarbazine, altretamine, or mitobronitol;

d) a DNA cross-linking agent such as cisplatin, carboplatin, nedaplatin, oxaliplatin, triplatin, tetranitrate or satraplatin;

e) a topoisomerase II inhibitor such as doxorubicin, epirubicin, aclarubicin, daunorubicin, idarubicin, amrubicin, pirarubicin, valrubicin or zorubicin, mitoxantrone or pixantrone;

f) a topoisomerase I inhibitor such as topotecan, camptothesin, irinotecan, rubitecan or belotecan;

g) an anti metabolite (pyrmidine analogue) such as 5-FU, capecitabine, tegafur, carmofur, floxuridine or cytarabine;

h) an anti metabolite (purine analogue) such as 6-thioguanine or mercaptopurine; or

i) a cytotoxic DNA alkylating agent.

The most preferred cytotoxic agent is temozolomide (TMZ).

For synergy between vector/prod rug/cytotoxic, it is necessary for the MMR pathway to become upregulated, and therefore administration protocol/dosage regimen is key.

As used herein, “cytotoxic therapy” and “prodrug therapy” means the cytotoxic and prodrug dosage regimens, courses of treatment. Those therapies are for a specified period of time. The vector, however, need only be administered once.

Preferably, the another cytotoxic agent therapy begins no later than 7 days after prodrug therapy has finished. More preferably, the cytotoxic agent therapy begins no later than 6, 5, 4, 3, 2 or 1 day after prodrug therapy has finished. Preferably, the cytotoxic agent therapy begins less than 1 day after prodrug therapy finishes.

For the avoidance of doubt, included within the scope of the invention is both the situation where cytotoxic therapy is started immediately after prodrug therapy has finished, and also the situation where cytotoxic therapy is started before the prodrug therapy has finished (i.e. there is a period of simultaneous administration.

The cytotoxic therapy and the prodrug therapy may be started at the same time. Although, preferably, the cytotoxic agent therapy begins no earlier than 2 days after prodrug therapy begins. This allows for most efficient administration as the cytotoxic and prodrug are only combined once the MMR pathway has been upregulated. This is the most efficient dosage regimen.

Preferably, the prodrug therapy and the another cytotoxic agent therapy overlaps. More preferably, the therapies overlap for at least 3 days. More preferably, they overlap for at least 7, 10, 14 or 18 days.

Preferably, the prodrug therapy lasts for from 10 to 20 days. More preferably, it lasts for from 11 to 19, 12 to 18 or 13 to 17 days. Preferably, it lasts for 14 days.

In a preferred embodiment, the prodrug therapy begins from 2 to 5 days after vector administration (gene transfer). More preferably, the prodrug therapy begins at 5 days after gene transfer.

Preferably, the another cytotoxic therapy should begin at the earliest at 2 days after starting prodrug therapy, and at the latest at 7 days after stopping prodrug therapy.

The another cytotoxic agent therapy should begin no earlier than simultaneously with the commencement of prodrug therapy. It will be appreciated that it is preferred for the another cytotoxic agent therapy to begin no earlier than 2 days after commencement of prodrug therapy.

The up-regulation of the MMR pathway is key to the invention. Therefore, it will be appreciated that the agent of the invention is useful in the treatment of a number of conditions. Examples of those conditions are cancer, actinic keratosis, pterygium diabetic retinopathy, atherosclerosis, asthma, chronic obstructive pulmonary disease, sarcoidosis, idiopathic pulmonary fibrosis, rheumatoid arthritis, pseudoexfoliation syndrome of the eye and Alzheimer's disease.

The most preferred therapy is of cancer. Preferably, the therapy is of a cancerous tumor, such as malignant glioma, or a tumor of the prostate. An agent of the invention may be used in the therapy of a cancer characterized by a normal or an impaired MMR pathway.

In a further preferred embodiment, an agent according to the present invention, when used to treat a cancerous tumor, also includes the administration of radiation. The radiation is preferably administered after the administration of the vector and the prodrug, and radiation therapy preferably starts at the same time as the cytotoxic chemotherapeutic agent (preferably, therapy is simultaneous).

The following Examples illustrate the present invention.

### EXAMPLE 1

A study was conducted concerning tumor growth rate in a rat glioma model. There were 6 patient groups. Details of agents administered and the dosage regimen are shown in Table 1 below.

TABLE 1

| G | Treatment | n | MRI | Tx | Gap1 | GCV | Gap2 | TMZ |
|---|---|---|---|---|---|---|---|---|
| 1 | Control | 7 | 0 | n/a | n/a | n/a | n/a | n/a |
| 2 | TMZ | 10 | 0 | n/a | n/a | n/a | n/a | 5-9 |
| 3 | AdHSVtk + | 18 | 0 | 1 | 4 | 5-11 | n/a | n/a |
| | GCV | | | | | | | |
| 4 | AdHSVtk + | 18 | 0 | 1 | 4 | 5-11 | 5 | 17-21 |
| | GCV + TMZ | | | | | | | |
| 5 | AdHSVtk + | 7 | 0 | 1 | 0 | 2-9 | 0 | 9-13 |
| | GCV + TMZ | | | | | | | |
| 6 | AdHSVtk + | 7 | 0 | 1 | 4 | 5-18 | n/a | 14-18 |
| | GCV + TMZ | | | | | | | |

Key:
G = Group number
MRI = Day number on which tumor was verified by Magnetic Resonance Imaging
Tx = Day number on which gene transfer was performed
Gap1 = Gap (number of days) between Tx and GCV
GCV = Day numbers on which ganciclovir was administered
Gap2 = Gap (number of days) between GCV and TMZ
TMZ = Day numbers on which temozolomide was administered

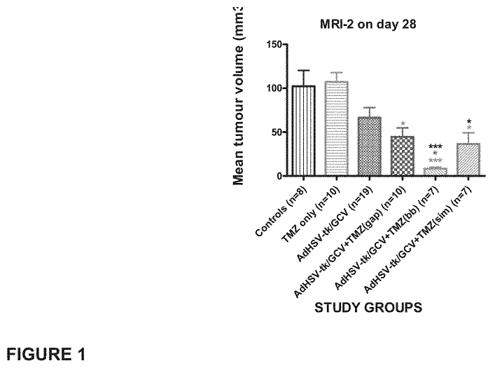

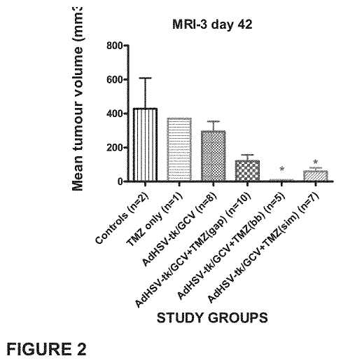

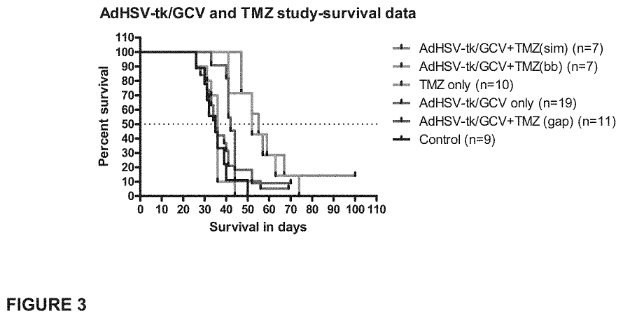

The results are shown in FIGS. 1 and 2. Group 5 shows the biggest decrease in tumor size.

### EXAMPLE 2

A second study was conducted in the rat glioma model concerning survival rates. The data (FIG. 2) show that Group 6 had the longest survival rate, closely followed by group 5. This partly led the inventors to devise the dosage regimen of the invention, as slight overlap of prodrug/cytotoxic therapy is beneficial

1. A method of treating cancer in a human patient, said method comprising:
diagnosing a cancer in a human patient,
administering to said human patient a viral gene therapy vector having a transgene,
wherein said transgene codes for thymidine kinase,
administering to said human patient ganciclovir, and
within about 30 days of said administration of said viral gene therapy vector, administering to said human patient a cytotoxic agent other than gancylovir, wherein said administration of said cytotoxic agent begins no earlier than 2 days after said administration of said gancyclovir begins.

2. The method of claim 1, wherein said administration of said cytotoxic agent and said administration of said gancyclovir overlap temporally.

3. The method of claim 2, wherein said administration of said cytotoxic agent and said administration of said gancyclovir overlap temporally for at least 3 days.

4. A method of treating cancer in a human patient, said method comprising:
diagnosing a cancer in a human patient,
administering to said human patient a viral gene therapy vector having a transgene,
wherein said transgene codes for thymidine kinase,
administering to said human patient ganciclovir,
wherein said administration of gancyclovir lasts for from about 10 to about 20 days, and
within about 30 days of said administration of said viral gene therapy vector, administering to said human patient a cytotoxic agent other than gancylovir.

5. In a method of treating cancer in an immunocompetent human patient by administering to said immunocompetent human patient a cytotoxic agent other than gancyclovir, the improvement comprising:
administering to said immunocompetent human patient a viral gene therapy vector, said administration of said viral gene therapy vector being within about 30 days of said administration of a cytotoxic agent other than gancyclovir, wherein said viral gene therapy vector is administered in an amount of about 3×10³ cfu.

6. A method of treating cancer in a human patient, said method comprising:
diagnosing a cancer in a human patient,
administering to said human patient a viral gene therapy vector comprising a virus modified to have a non-native nucleic acid sequence which is not native to the virus wild-type genome, the non-native nucleic acid sequence coding for a non-native polypeptide which is not expressed by the virus wild-type genome, and
within about 30 days of said administration of said viral gene therapy vector, administering to said human patient a cytotoxic agent other than gancylovir.

7. The method of claim 6, wherein said non-native nucleic acid sequence codes for thymidine kinase.

8. The method of claim 7, further comprising:
administering to said human patient gancyclovir.

9. The method of claim 8, wherein said administration of said cytotoxic agent begins no earlier than 2 days after said administration of said gancyclovir begins.

10. The method of claim 9, wherein said administration of said cytotoxic agent and said administration of said gancyclovir overlap temporally.

11. The method of claim 10, wherein said administration of said cytotoxic agent and said administration of said gancyclovir overlap temporally for at least 3 days.

12. The method of claim 8, wherein said administration of gancyclovir lasts for from about 10 to about 20 days.

13. The method of claim 6, wherein said administration of said cytotoxic agent other than gancylovir lasts for up to 50 days.

14. The method of claim 6, wherein said cancer is selected from the group consisting of: brain cancer, prostate cancer and bladder cancer.

15. The method of claim 6, further comprising:
resecting cancer cells from said human patient, to form a cavity, said cavity bounded by a cavity wall.

16. The method of claim 15, wherein said viral gene therapy vector is administered into tissue that forms said cavity wall.

17. The method of claim 16, wherein said viral gene therapy vector is administered into said tissue that forms said cavity wall, to a depth of approximately 1 cm.

18. The method of claim 6, further comprising:
administering to said human patient radiotherapy.

19. The method of claim 6, wherein said viral gene therapy vector is derived from an adenovirus or a lentivirus.

20. The method of claim 6, wherein said cytotoxic agent is selected from the group consisting of: chloroethylating agent, non-classical alkylating agent, methylating triazine, DNA cross-linking agent, topoisomerase inhibitor, pyridine analogue, antifolate and DNA alkylating agent.

21. The method of claim 6, wherein said cytotoxic agent comprises a DNA cross-linking agent selected from the group consisting of:
cisplatin, carboplatin, nedaplatin, oxaliplatin, triplatin, tetranitrate and satraplatin.

22. The method of claim 6, wherein said cytotoxic agent comprises pemetrexed.

23. The method of claim 6, wherein said cytotoxic agent comprises lomustine.

24. In a method of treating cancer in an immunocompetent human patient by administering to said immunocompetent human patient a cytotoxic agent other than gancyclovir, the improvement comprising:
administering to said immunocompetent human patient a viral gene therapy vector comprising a virus modified to have a non-native nucleic acid sequence which is not native to the virus wild-type genome, the non-native nucleic acid sequence coding for a non-native polypeptide which is not expressed by the virus wild-type genome,
said administration of said viral gene therapy vector being within about 30 days of said administration of a cytotoxic agent other than gancyclovir.

25. The method of claim 24, wherein said viral gene therapy vector is administered in an amount of about 3×10³ cfu.

26. The method of claim 25, further comprising: resecting at least part of said brain cancer.

27. The method of claim 26, wherein said resecting forms a cavity and wherein said cavity has a cavity wall, and wherein said administration of said viral vector comprises administration to the wall of the cavity formed by the resecting.

28. The method of claim 24, wherein said viral vector comprises adenovirus.

29. The method of claim 24, wherein said viral vector comprises a thymidine kinase transgene, and wherein said method of treatment further comprises administering to said human patient ganciclovir or an analogue thereof.

30. The method of claim 24, wherein said cancer is selected from the group consisting of: bladder cancer, prostate cancer, malignant glioma and anaplastic astrocytoma.

31. The method of claim 24, further comprising administering to said human patient focal radiotherapy.

32. The method of claim 29, wherein said administration of said ganciclovir or analogue thereof lasts for from about 10 to about 20 days.

33. The method of claim 29, further comprising administering to said human patient temozolomide, wherein said administration of said temozolomide lasts for not more than about 50 days.

34. The method of claim 33, wherein said administration of temozomide begins not earlier than about 2 days after said administration of ganciclovir or an analogue thereof.

35. The method of claim 33, wherein said administration of said temozomide begins not later than about 7 days after said administration of said ganciclovir or analogue thereof.

36. The method of claim 35, wherein said administration of said temozomide overlaps said administration of said ganciclovir or analogue thereof.

37. The method of claim 36, wherein said overlap is for at least 3 days.

38. A kit comprising a viral vector comprising a virus modified to have a non-native nucleic acid sequence which is not native to the virus wild-type genome, the non-native nucleic acid sequence coding for a non-native polypeptide which is not expressed by the virus wild-type genome and temozolomide, said viral vector and said temozolomide present in an amount effective to treat brain cancer in a human patient.

39. The kit of claim 38, wherein said viral vector comprises a thymidine kinase transgene.
